(19) United States
(12) Patent Application Publication
(10) Pub. No.: US 2025/0387274 A1
(43) Pub. Date: Dec. 25, 2025

(54) ENVIRONMENTALLY FRIENDLY CLOTH DIAPER HAVING INTERCHANGABLE WETNESS INDICATOR

(71) Applicant: SHIH-WHY LIN, Taipei City (TW)

(72) Inventor: SHIH-WHY LIN, Taipei City (TW)

(21) Appl. No.: 19/304,700

(22) Filed: Aug. 20, 2025

Related U.S. Application Data
(63) Continuation-in-part of application No. 18/108,909, filed on Feb. 13, 2023.

(51) Int. Cl. A61F 13/42; A61F 13/15; A61F 13/62

(52) U.S. Cl. CPC A61F 13/42; A61F 13/15268; A61F 13/62; A61F 2013/15276; A61F 2013/422; A61F 2013/426; A61F 2013/429

(57) ABSTRACT

An environmentally friendly cloth diaper having an interchangeable wetness indicator is provided. The environmentally friendly cloth diaper includes a covering body and at least one wetness indicator. The covering body has an inner side layer, an outer side layer, and an absorption layer. The wetness indicator is interchangeably disposed on the covering body and is exposed from the outer side layer of the covering body, and urine absorbed in the absorption layer is able to be transported to the wetness indicator so that the color of the wetness indicator is changed.

### CROSS-REFERENCE TO RELATED PATENT APPLICATION

This application is a Continuation-In-Part of the U.S. application Ser. No. 18/108,909, filed on Feb. 13, 2023, and entitled “ENVIRONMENTALLY FRIENDLY CLOTH DIAPER HAVING INTERCHANGEABLE WETNESS INDICATOR,” now pending. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an environmentally friendly cloth diaper having an interchangeable wetness indicator, and more particularly to an environmentally friendly cloth diaper that is made of cloth and has a display function.

### BACKGROUND OF THE DISCLOSURE

Diapers are usually worn by infants, the elderly, and patients that are under care and may be disabled or unable to complete the normal process of urinating or defecating on their own. The diapers are then checked either regularly or irregularly to see if the diapers have absorbed too much urine or if a foul odor indicative of defecation is detected, so as to determine whether or not to change the diapers. Cloth diapers are mainly made of fabrics (such as a knitted fabric) and can be used repeatedly, such as to better comply with requirements regarding environmental protection.

Existing cloth diapers have a wetness indicating function (such as one disclosed in Taiwan Patent Publication No. TW M591863), in which a wetness indicator can be disposed on a covering body, and urine absorbed in the absorption layer can be transported to the wetness indicator, causing a color of the wetness indicator to be changed. Therefore, when the diaper becomes wet from the urine, a change in the color of the wetness indicator will be displayed immediately, and the caregiver can be reminded to change the cloth diaper in time, so as to prevent discomfort of the patient caused by negligence of changing the cloth diaper. However, in the abovementioned environmentally friendly cloth diaper having the wetness indicating function, the wetness indicator is fixed on the cover body, which causes inconvenience in assembly. Moreover, the wetness indicator cannot be replaced with a new one after being damaged, which can lead to a total loss of the wetness indicating function.

### SUMMARY OF THE DISCLOSURE

In response to the above-referenced technical inadequacies, the present disclosure provides an environmentally friendly cloth diaper having an interchangeable wetness indicator. The wetness indicator is interchangeably disposed to facilitate the assembly and removal of the wetness indicator, such that the wetness indicator can be changed easily when the wetness indicator is damaged.

In one aspect, the present disclosure provides an environmentally friendly cloth diaper. The environmentally friendly cloth diaper includes a covering body, a wetness indicator, and a retaining layer. The covering body has an outer side layer, and the outer side layer has a through hole. The wetness indicator is disposed at a position corresponding to the through hole. The wetness indicator includes a liquid-permeable color layer, a color-changeable layer, and a transparent protective layer. The color-changeable layer is made of humidity-sensitive ink and sprayed on the liquid-permeable color layer. The color-changeable layer is disposed between the liquid-permeable color layer and the transparent protective layer. The transparent protective layer faces the through hole. The wetness indicator is interchangeably disposed on the covering body, and the wetness indicator is exposed from the outer side layer of the covering body via the through hole. The retaining layer is disposed on an inner side of the outer side layer of the covering body to defined an accommodating space between the retaining layer and the outer side layer to receive the wetness indicator. The retaining layer is liquid-permeable, and the retaining layer faces the liquid-permeable color layer of the wetness indicator. When the retaining layer is in contact with a liquid, the liquid permeates the liquid-permeable color layer and contacts the color-changeable layer. The humidity-sensitive ink senses a humidity and the color-changeable layer is changed to be transparent, and the color of the liquid-permeable color layer is not shielded by the color-changeable layer, so that a color of the liquid-permeable color layer is appeared through the color-changeable layer, the transparent protective layer and the through hole of the outer side layer.

Therefore, in the environmentally friendly cloth diaper having the interchangeable wetness indicator of the present disclosure, the environmentally friendly cloth diaper includes a covering body and at least one wetness indicator. The wetness indicator is interchangeably disposed on the covering body and is washable, the wetness indicator is exposed from the outer side layer of the covering body, and urine absorbed in the absorption layer is able to be transported to the wetness indicator so that the color of the wetness indicator is changed. Therefore, the color change of the wetness indicator can be utilized to determine a wetness of the cloth diaper, which can be displayed immediately, such that the caregiver can be reminded to change the cloth diaper in time. Moreover, the wetness indicator is interchangeably disposed on the covering body, such that the wetness indicator can be changed easily when the wetness indicator is damaged.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of an environmentally friendly cloth diaper having an interchangeable wetness indicator according to a first embodiment of the present disclosure;

FIG. 2 is a schematic perspective view of the environmentally friendly cloth diaper in a worn state according to the first embodiment of the present disclosure;

FIG. 3 is a rear view of the environmentally friendly cloth diaper according to the first embodiment of the present disclosure;

FIG. 4 is a cross-sectional view of the environmentally friendly cloth diaper according to the first embodiment of the present disclosure;

FIG. 5 is a schematic perspective view of an internal structure of the environmentally friendly cloth diaper according to the first embodiment of the present disclosure;

FIG. 6 is a front view of an environmentally friendly cloth diaper according to a second embodiment of the present disclosure;

FIG. 7 is a rear view of the environmentally friendly cloth diaper according to the second embodiment of the present disclosure;

FIG. 8 is a schematic perspective view of an environmentally friendly cloth diaper according to a third another embodiment of the present disclosure;

FIG. 9 is a top view of the environmentally friendly cloth diaper according to the third embodiment of the present disclosure; and

FIG. 10 is a top view of an environmentally friendly cloth diaper according to a fourth embodiment of the present disclosure.

FIG. 11 is an partial exploded view of an environmentally friendly cloth diaper according to a fifth embodiment of the present disclosure.

FIG. 12 is an exploded view of the wetness indicator of the present disclosure.

FIG. 13 is an partial cross-sectional of the environmentally friendly cloth diaper of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

### First Embodiment

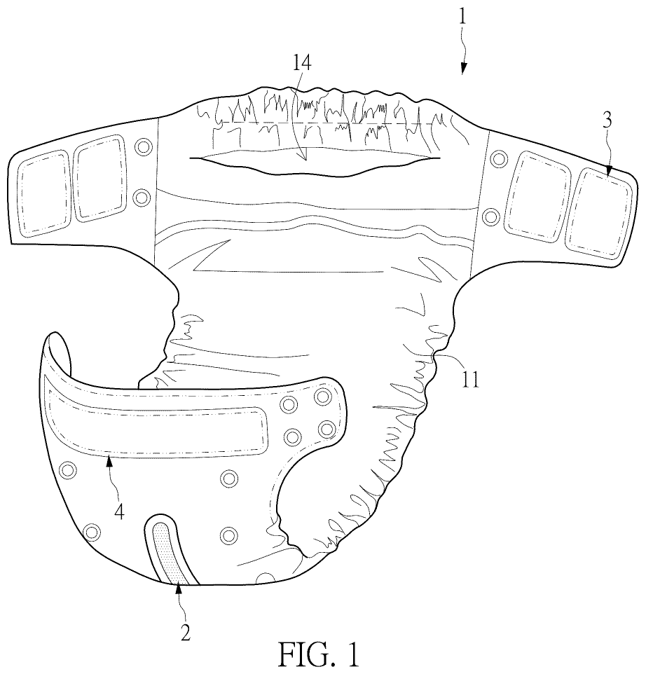

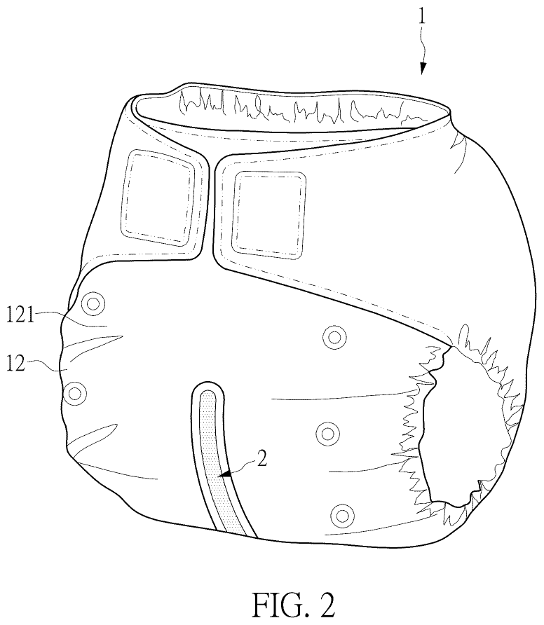

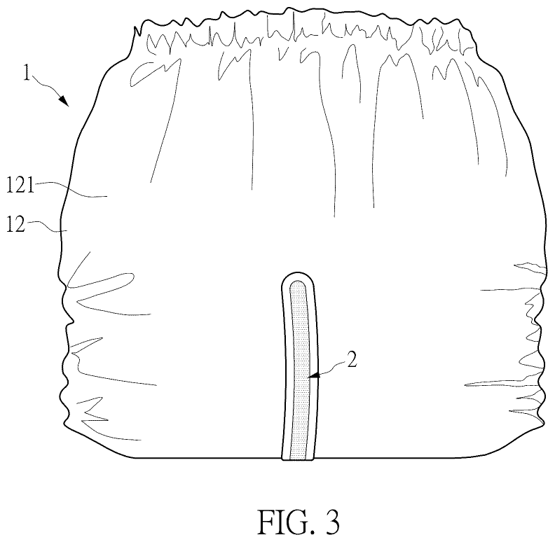

An environmentally friendly cloth diaper having an interchangeable wetness indicator is provided in the present disclosure. The environmentally friendly cloth diaper can be worn on a human body, that is, the environmentally friendly cloth diaper can be an infant diaper or an adult diaper. The environmentally friendly cloth diaper can also be worn on pets such as dogs or cats, that is, the environmentally friendly cloth diaper can be a pet diaper. The environmentally friendly cloth diaper is made of fabrics (such as a knitted fabric). Referring to FIG. 1 to FIG. 4, the environmentally friendly cloth diaper includes a covering body 1 and at least one wetness indicator 2.

The covering body 1 has an inner side layer 11, an outer side layer 12, and an absorption layer 13. The inner side layer 11 and the outer side layer 12 are located at two surfaces of the covering body 1 that are opposite to each other. The inner side layer 11 can face toward a torso (e.g., a lower torso) of a wearer, and the inner side layer 11 is liquid-permeable. The outer side layer 12 can face away from the torso of the wearer, and a waterproof coating layer 121 can be disposed on one side of the outer side layer 12, so that the outer side layer 12 is liquid-impermeable and prevents urine in the absorption layer 13 from leaking outward. The absorption layer 13 can be sandwiched between the inner side layer 11 and the outer side layer 12 or disposed on the inner side layer 11, and a manner in which the absorption layer 13 is disposed is not limited thereto. The absorption layer 13 is able to absorb liquid, so that when the covering body 1 is worn by the wearer, the urine can be absorbed into the absorption layer 13 through the inner side layer 11, so that the urine can be stored in the absorption layer 13.

Furthermore, two ends of the covering body can respectively have a first fixing portion 3 and a second fixing portion 4 disposed thereon, and the first fixing portion 3 and the second fixing portion 4 can be components of corresponding hook-and-loop fasteners or buckles. When the covering body 1 is worn by the wearer, the first fixing portion 3 and the second fixing portion 4 can be engaged to each other, so that the covering body 1 is stably fixed on the body of the wearer. A structure or form of the aforementioned covering body 1 is not limited in the present disclosure, and can be changed according to different types of the environmentally friendly cloth diaper or different wearers.

The wetness indicator 2 is a structure (e.g., a coating layer) that changes color when coming in contact with a liquid, and the wetness indicator 2 includes a coating that changes color when coming in contact with the liquid. The wetness indicator 2 is interchangeably disposed on the covering body 1, and the wetness indicator 2 is exposed from the outer side layer 12 of the covering body 1. A quantity of the wetness indicator 2 can be one or more than one, and the wetness indicator 2 can be in a shape of a pattern, a text, or a number, and is not limited thereto. In this embodiment, the wetness indicator 2 is strip shaped, and the wetness indicator 2 extends along a longitudinal direction of the covering body 1. The wetness indicator 2 is extended to be adjacent to two ends of the covering body 1, so as to have a wider range for display.

In this embodiment, the wetness indicator 2 is located at middle of the covering body 1, and is adjacent to a bottom of the covering body 1. Quantities and locations in which the wetness indicator 2 is disposed are not limited thereto, and can be changed according to requirements. A through hole 122 can be formed at a position on the outer side layer 12 and the waterproof coating layer 121 of the covering body 1 corresponding to the wetness indicator 2, so that the wetness indicator 2 can be exposed from the outer side layer 12 of the covering body 1 via the through hole 122, and at least one waterproof layer 123 can be disposed at the through hole 122. A quantity of the waterproof layer 123 can be one or two, and one waterproof layer 123 is disposed in the present embodiment. Preferably, two waterproof layers 123 are disposed, and the two waterproof layers 123 are correspondingly adhered to each other at an inner side and an outer side of a position at the through hole 122, so as to provide an improved waterproofing effect.

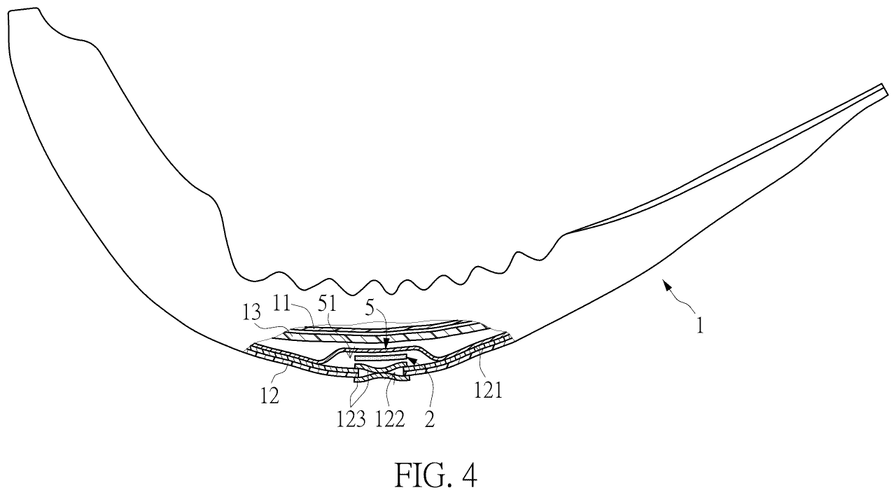

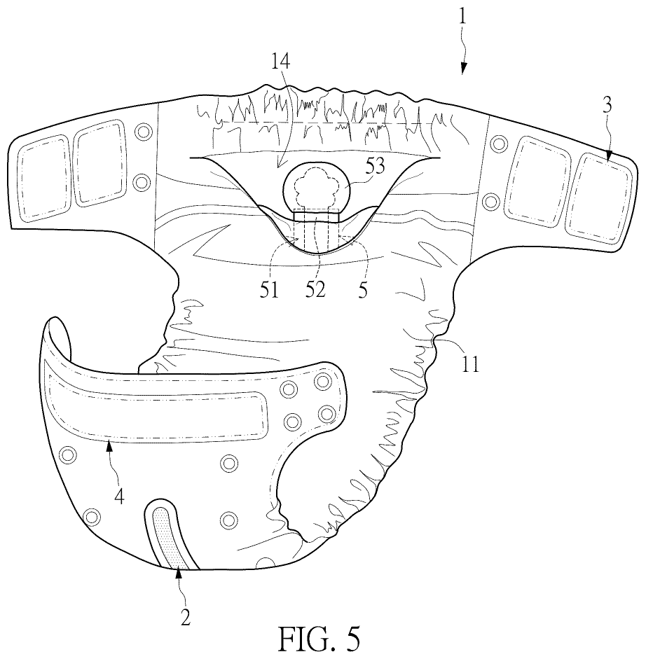

In this embodiment, a bag 5 is disposed on the covering body 1 (as shown in FIG. 4 and FIG. 5), the bag 5 is disposed on an inner side of the outer side layer 12 of the covering body 1, and the bag 5 is liquid-permeable, such as cloth. An accommodating space 51 is formed in the bag 5, and the accommodating space 51 is adjacent to the outer side layer 12. One end (e.g., an upper end) of the bag 5 has a bag opening 52 that is in spatial communication with the accommodating space 51. The wetness indicator 2 can be inserted into the accommodating space 51 through the bag opening 52, so that the wetness indicator 2 can be disposed in the bag 5, thereby allowing the wetness indicator 2 to be interchangeably disposed on the covering body 1. In this embodiment, the wetness indicator 2 is positioned using the bag 5, so that the wetness indicator 2 can be quickly disposed on the covering body 1, and the wetness indicator 2 can be changed easily. In another practical embodiment, the bag 5 can be a retaining layer with a single layer.

In this embodiment, the bag opening 52 of the bag 5 can have a bag cover 53 disposed thereon, and the bag cover 53 is covered on the bag opening 52 and is able to be lifted to uncover the bag opening 52. By the bag cover 53 being covered on the bag opening 52, the wetness indicator 2 can be prevented from escaping the accommodating space 51 and can be stably disposed at the accommodating space 51. Preferably, one end of the wetness indicator 2 extends out from the bag opening 52, and the bag cover 53 is covered on the one end of the wetness indicator 2 that extends out from the bag opening 52, so that the wetness indicator 2 can be stably positioned by using the bag 5 and the bag cover 53.

The urine contained in the absorption layer 13 can leak into the wetness indicator 2 through the bag 5. The wetness indicator 2 can change color when coming in contact with a liquid, for example, the wetness indicator 2 can display a first color when the wetness indicator 2 is dry, and display a second color when the wetness indicator 2 is wet. Therefore, the wetness indicator 2 can change color to indicate wetness.

In this embodiment, an opening 14 (as shown in FIG. 1) is further formed between the inner side layer 11 and the outer side layer 12 of the covering body 1, so that the absorption layer 13 can be placed between the inner side layer 11 and the outer side layer 12 through the opening 14. Moreover, the opening 14 allows the wetness indicator 2 to be conveniently placed in the covering body 1, and the wetness indicator 2 can be placed in the bag 5 through the opening 14.

### Second Embodiment

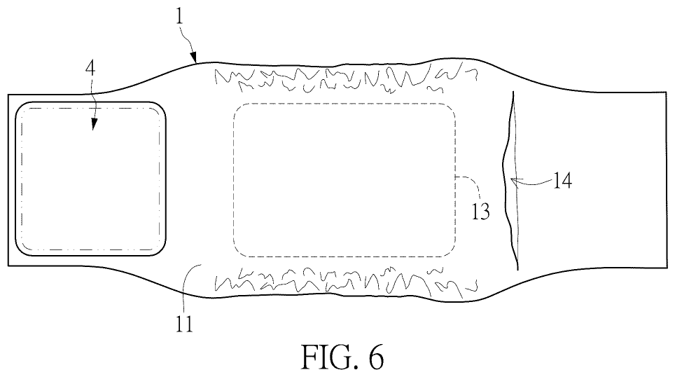

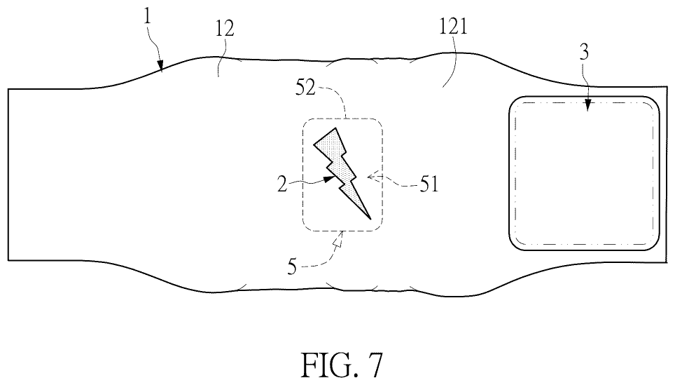

Referring to FIG. 6 and FIG. 7, an environmentally friendly cloth diaper having an interchangeable wetness indicator is provided in this embodiment, and the environmentally friendly cloth diaper can be worn by pets such as dogs or cats. Specifically, the environmentally friendly cloth diaper is worn by male dogs in this embodiment. The environmentally friendly cloth diaper includes a covering body 1 and at least one wetness indicator 2, and a shape of the covering body 1 can be adequately modified according to a body size of the pet. The covering body 1 is substantially in a shape of an elongated belt, and has an inner side layer 11, an outer side layer 12, and an absorption layer 13. The inner side layer 11 and the outer side layer 12 are located at two surfaces of the covering body 1 that are opposite to each other. The inner side layer 11 is liquid-permeable, and a waterproof coating layer 121 can be disposed on one side of the outer side layer 12, so that the outer side layer 12 is liquid-impermeable. The absorption layer 13 can be sandwiched between the inner side layer 11 and the outer side layer 12 or disposed on the inner side layer 11, and the absorption layer 13 is able to absorb liquid. The wetness indicator 2 changes color when coming in contact with a liquid, the wetness indicator 2 is interchangeably disposed on the covering body 1, and the wetness indicator 2 is exposed from the outer side layer 12 of the covering body 1. In this embodiment, a bag 5 is disposed on the covering body 1, and the bag 5 is disposed on an inner side of the outer side layer 12 of the covering body 1. An accommodating space 51 is formed in the bag 5, and the accommodating space 51 is adjacent to the outer side layer 12. One end of the bag 5 has a bag opening 52 that is in spatial communication with the accommodating space 51. The wetness indicator 2 can be inserted into the accommodating space 51 through the bag opening 52, so that the wetness indicator 2 can be disposed in the bag 5, thereby allowing the wetness indicator 2 to be interchangeably disposed on the covering body 1. The urine stored in the absorption layer 13 can be transported to the wetness indicator 2, allowing the wetness indicator 2 to change color.

### Third Embodiment

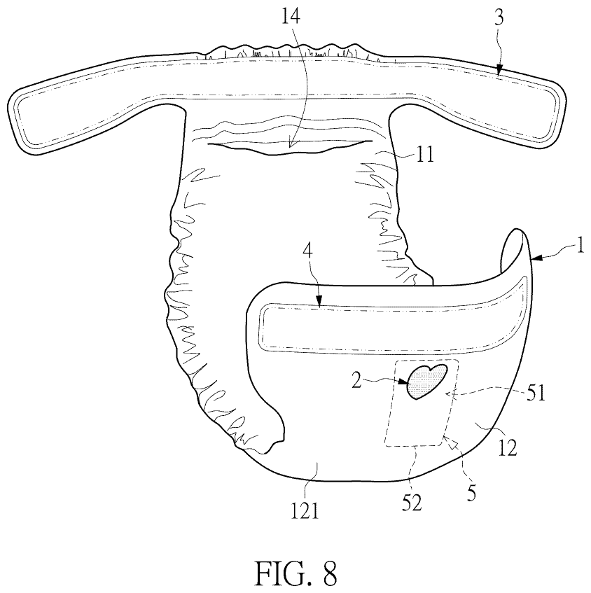

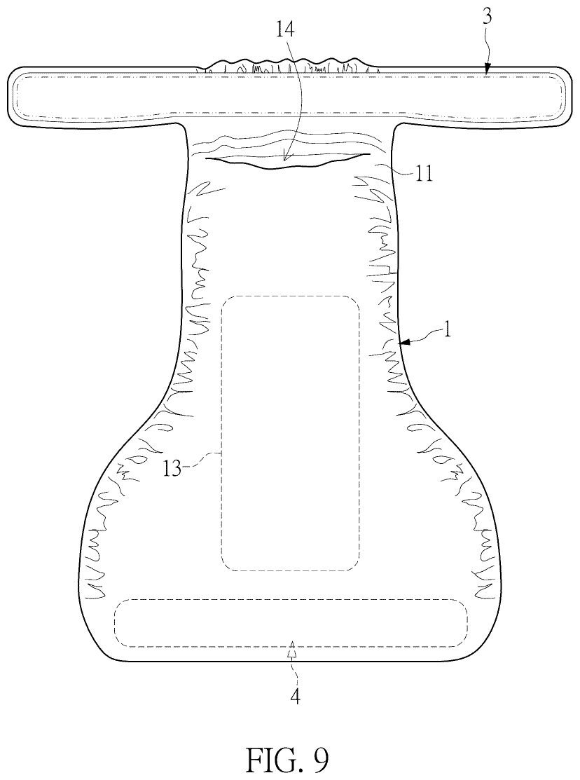

Referring to FIG. 8 and FIG. 9, an environmentally friendly cloth diaper having an interchangeable wetness indicator is provided in this embodiment, and the environmentally friendly cloth diaper can be worn by pets such as dogs or cats. Specifically, the environmentally friendly cloth diaper is worn by female dogs in this embodiment. The environmentally friendly cloth diaper includes a covering body 1 and at least one wetness indicator 2, and a shape of the covering body 1 can be adequately modified according to a body size of the pet. The covering body 1 is substantially in a shape of an elongated belt, and has an inner side layer 11, an outer side layer 12, and an absorption layer 13. The inner side layer 11 and the outer side layer 12 are located at two surfaces of the covering body 1 that are opposite to each other. The inner side layer 11 is liquid-permeable, and a waterproof coating layer 121 can be disposed on one side of the outer side layer 12, so that the outer side layer 12 is liquid-impermeable. The absorption layer 13 can be sandwiched between the inner side layer 11 and the outer side layer 12 or disposed on the inner side layer 11, and the absorption layer 13 is able to absorb liquid. The wetness indicator 2 changes color when coming in contact with a liquid, the wetness indicator 2 is interchangeably disposed on the covering body 1, and the wetness indicator 2 is exposed from the outer side layer 12 of the covering body 1. In this embodiment, a bag 5 is disposed on the covering body 1, and the bag 5 is disposed on an inner side of the outer side layer 12 of the covering body 1. An accommodating space 51 is formed in the bag 5, and the accommodating space 51 is adjacent to the outer side layer 12. One end of the bag 5 has a bag opening 52 that is in spatial communication with the accommodating space 51. The wetness indicator 2 can be inserted into the accommodating space 51 through the bag opening 52, so that the wetness indicator 2 can be disposed in the bag 5, thereby allowing the wetness indicator 2 to be interchangeably disposed on the covering body 1. The urine stored in the absorption layer 13 can be transported to the wetness indicator 2, allowing the wetness indicator 2 to change color.

### Fourth Embodiment

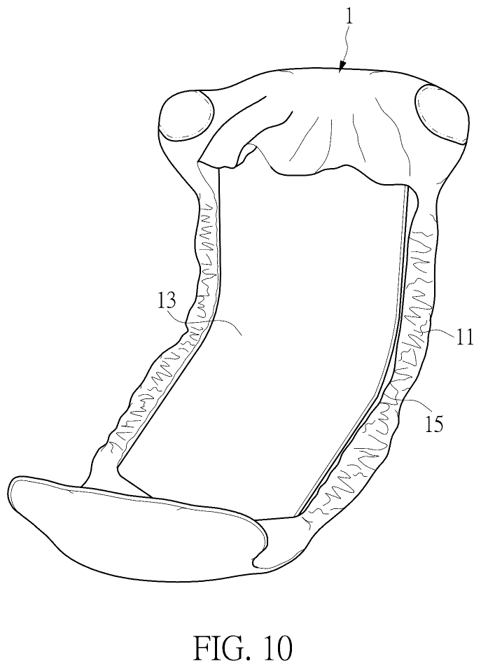

Referring to FIG. 10, an environmentally friendly cloth diaper having an interchangeable wetness indicator is provided in this embodiment, and the environmentally friendly cloth diaper is a hybrid cloth diaper. A pocket (or stay-put layer) 15 is disposed on the inner side layer 11 of the covering body 1, and the absorption layer 13 can be placed in the pocket 15, so that the absorption layer 13 is disposed on the inner side layer 11. The absorption layer 13 is externally disposed onto the covering body 1. The wetness indicator disclosed in the aforementioned embodiments can also be disposed on the covering body 1 (as shown in FIG. 1 to FIG. 5, FIG. 7, and FIG. 8).

### Fifth Embodiment

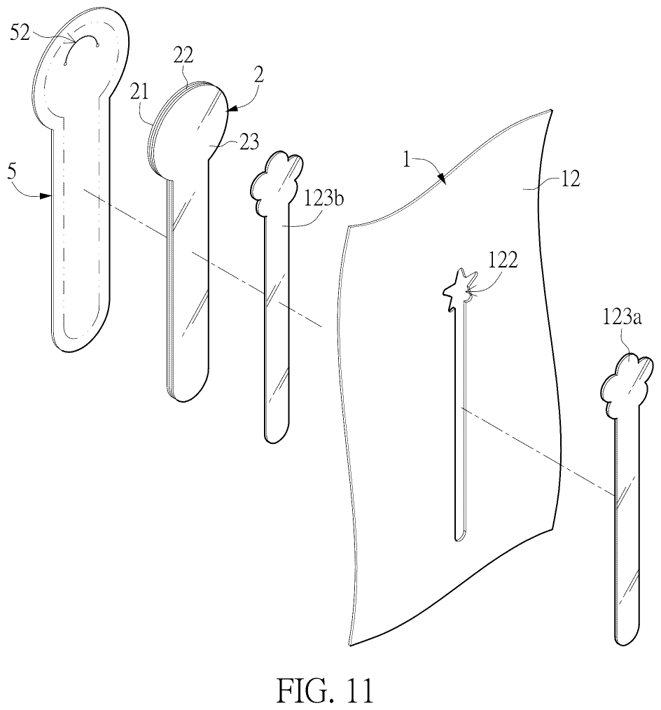

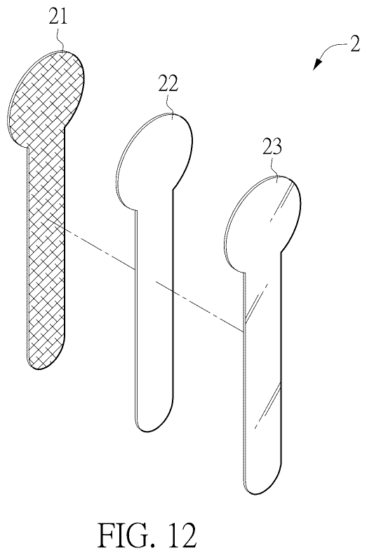

Referring to FIGS. 11 to 13, an environmentally friendly cloth diaper having an interchangeable wetness indicator is provided in this embodiment. The environmentally friendly cloth diaper includes a covering body 1, a wetness indicator 2, and a retaining layer 5.

The covering body 1 can be the same as the above embodiments. For example, as shown in FIG. 4, the covering body 1 includes the inner side layer 11, the outer side layer 12, and the absorption layer 13. The outer side layer 12 has a through hole 122. In this embodiment, the through hole 122 has a shape of a star and a strip connected to the star.

The wetness indicator 2 is disposed at a position corresponding to the through hole 122. The wetness indicator 2 includes a liquid-permeable color layer 21, a color-changeable layer 22, and a transparent protective layer 23. The liquid-permeable color layer 21 can be, for example, a cloth, and dyed with a bright color, that is washable. The color-changeable layer 22 is made of humidity-sensitive ink and sprayed on the liquid-permeable color layer 21. The color-changeable layer 22 is disposed between the liquid-permeable color layer 21 and the transparent protective layer 23. The color-changeable layer 22 is used to shield a color of the liquid-permeable color layer 21 in a dry state. The transparent protective layer 23 is covered on the color-changeable layer 22 and faced the through hole 122, so as to protect the color-changeable layer 22. Thus, the wetness indicator is washable. The transparent protective layer 23 can protect the color-changeable layer 22 from falling off during the washing process. The wetness indicator 2 is interchangeably disposed on the covering body 1, and the wetness indicator 2 is exposed from the outer side layer 12 of the covering body 1 via the through hole 122.

The retaining layer 5 is disposed on an inner side of the outer side layer 12 of the covering body 1 to defined an accommodating space between the retaining layer 5 and the outer side layer 12 to receive the wetness indicator 2. The retaining layer 5 is liquid-permeable, and the retaining layer 5 faces the liquid-permeable color layer 21 of the wetness indicator 2. The retaining layer 5 can be, for example, cloth.

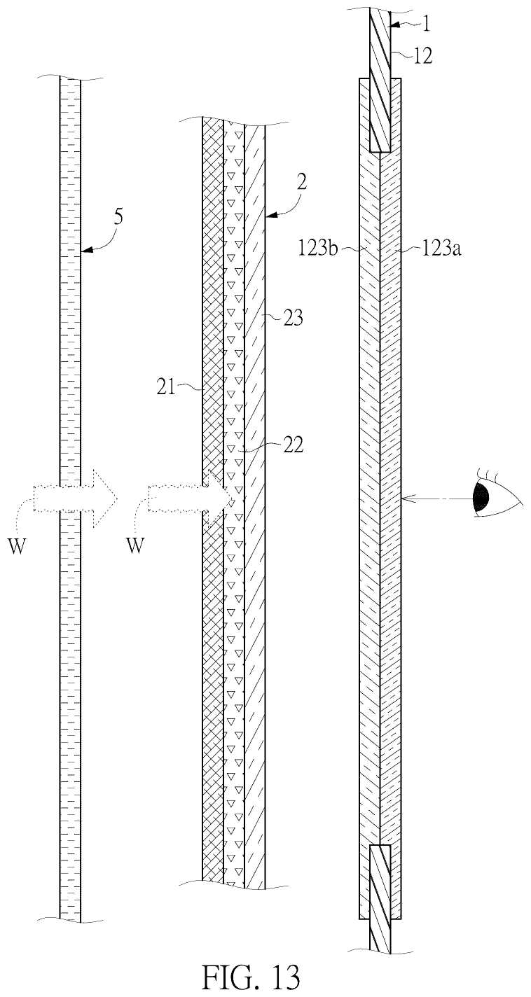

Referring to FIG. 13, when the retaining layer 5 is in contact with a liquid W, the liquid W permeates the liquid-permeable color layer 21 and contacts the color-changeable layer 22. The humidity-sensitive ink is able to sense a humidity change and changes to be transparent, and the liquid-permeable color layer 21 is unshielded by the color-changeable layer 22, so that a color of the liquid-permeable color layer 21 is appeared through the transparent color-changeable layer 22, the transparent protective layer 23 and the through hole 122 of the outer side layer 12.

In a practical embodiment, the humidity-sensitive ink of the color-changeable layer 22 includes a humidity-sensitive material, a solvent, and an adhesive. The humidity-sensitive material includes white carbon black and acrylic emulsion. The color-changeable layer 22 is white and is able to become transparent, when the humidity-sensitive ink comes into contact with liquid.

Acrylic acid is a unsaturated carboxylic acid, and the chemical formula is C₃H₄O₂. Acrylic emulsion is a water-dispersible high molecular polymer made from acrylic ester monomers through emulsion polymerization. Its main characteristics are water dispersibility and film-forming properties. Regarding water dispersibility, acrylic emulsion is water-dispersible and easily dissolves in water. Regarding film-forming properties, acrylic emulsion can form a transparent film with a certain hardness after drying.

The white carbon black also known as silica, that is a synthetic hydrated silicon dioxide with the molecular formula mSiO2·nH2O. It appears as a white, fluffy powder. It absorbs moisture from the air and aggregates into fine particles.

The color-changing mechanism behind the reversible, moisture-sensitive color-changing ink is that they are composed of a porous material. When light passes through this porous, irregular structure in its dry state, it scatters light, creating a white, opaque, and masking effect. Water entering the pores increases light refraction, turning the material transparent and revealing the shielded pattern or color. Once the water evaporates, the ink returns to its opaque white state.

The retaining layer 5 has a fitting opening 52 that is in spatial communication with the accommodating space, and the wetness indicator 2 can be inserted into the accommodating space through the fitting opening 52. The retaining layer 5 can be the same as the above embodiments.

Referring to FIG. 11, an outer waterproof layer 123a is disposed on an outer side of the outer side layer 12, and an inner waterproofing layer 123b is disposed on an inner side of the outer side layer 12. The outer waterproofing layer 123a and the inner waterproofing layer 123b are attached to each other to seal the through hole 122. Therefore, an user can observe the wetness indicator 2 through the through hole 122 of the outer side layer 12.

### BENEFICIAL EFFECTS OF THE EMBODIMENTS

In conclusion, in the environmentally friendly cloth diaper having the interchangeable wetness indicator of the present disclosure, the environmentally friendly cloth diaper includes a covering body and at least one wetness indicator. Not only the covering body, but also the wetness indicator is reusable and washable, since the wetness indicator has three-layers structure. The wetness indicator is interchangeably disposed on the covering body, the wetness indicator is exposed from the outer side layer of the covering body, and urine absorbed in the absorption layer is able to be transported to the wetness indicator so that the color of the wetness indicator is changed. Therefore, the color change of the wetness indicator can be utilized to determine a wetness of the cloth diaper, which can be displayed immediately, such that the caregiver can be reminded to change the cloth diaper in time. Moreover, the wetness indicator is interchangeably disposed on the covering body, such that the wetness indicator can be changed easily when the wetness indicator is damaged.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An environmentally friendly cloth diaper, comprising:
a covering body having an outer side layer, the outer side layer having a through hole;
a wetness indicator, disposed at a position corresponding to the through hole, wherein the wetness indicator including a liquid-permeable color layer, a color-changeable layer, and a transparent protective layer, the color-changeable layer is made of humidity-sensitive ink and sprayed on the liquid-permeable color layer, the color-changeable layer is disposed between the liquid-permeable color layer and the transparent protective layer, the transparent protective layer faces the through hole; wherein the wetness indicator is interchangeably disposed on the covering body, and the wetness indicator is exposed from the outer side layer of the covering body via the through hole; and
a retaining layer, disposed on an inner side of the outer side layer of the covering body to defined an accommodating space between the retaining layer and the outer side layer to receive the wetness indicator, wherein the retaining layer is liquid-permeable, and the retaining layer faces the liquid-permeable color layer of the wetness indicator;
wherein, when the retaining layer is in contact with a liquid, the liquid permeates the liquid-permeable color layer and contacts the color-changeable layer, the humidity-sensitive ink senses a humidity and the color-changeable layer is changed to be transparent, and the color of the liquid-permeable color layer is unshielded by the color-changeable layer, so that a color of the liquid-permeable color layer is appeared through the color-changeable layer, the transparent protective layer and the through hole of the outer side layer.

2. The environmentally friendly cloth diaper according to claim 1, wherein the covering body further includes an inner side layer, and an absorption layer, the inner side layer and the outer side layer are located at two surfaces of the covering body that are opposite to each other, wherein the absorption layer is sandwiched between the inner side layer and the outer side layer or disposed on the inner side layer, and the absorption layer is capable of absorbing liquid, and the liquid absorbed in the absorption layer is capable of being transported to the wetness indicator.

3. The environmentally friendly cloth diaper according to claim 1, wherein the retaining layer has a fitting opening that is in spatial communication with the accommodating space, and the wetness indicator is inserted into the accommodating space through the fitting opening.

4. The environmentally friendly cloth diaper according to claim 1, wherein the humidity-sensitive ink includes a humidity-sensitive material, a solvent, and an adhesive, the humidity-sensitive material includes white carbon black and acrylic emulsion, the humidity-sensitive ink is white, wherein, when the humidity-sensitive ink comes into contact with liquid, the humidity-sensitive ink becomes to be transparent.

5. The environmentally friendly cloth diaper according to claim 1, wherein the wetness indicator is strip shaped, the wetness indicator extends along a longitudinal direction of the covering body, and the wetness indicator is adjacent to two ends of the covering body.

6. The environmentally friendly cloth diaper according to claim 1, wherein the environmentally friendly cloth diaper is an infant diaper or an adult diaper.

7. The environmentally friendly cloth diaper according to claim 1, wherein the environmentally friendly cloth diaper is a pet diaper.

8. The environmentally friendly cloth diaper according to claim 1, wherein an outer waterproof layer is disposed on an outer side of the outer side layer, and an inner waterproofing layer is disposed on an inner side of the outer side layer, the outer waterproofing layer and the inner waterproofing layer are attached to each other to seal the through hole.
